(19) United States
(12) Patent Application Publication
(10) Pub. No.: US 2022/0356505 A1
(43) Pub. Date: Nov. 10, 2022

(54) DEVICES AND METHODS FOR THE DETECTION OF BACTERIA

(71) Applicant: Transformative Technologies, Baltimore, MD (US)

(72) Inventors: Lawrence SILVER, Westbury, NY (US); Lawrence LOOMIS, Columbia, MD (US)

(73) Assignee: Transformative Technologies, Baltimore, MD (US)

(21) Appl. No.: 17/623,162

(22) PCT Filed: Jun. 26, 2020
(86) PCT No.: PCT/US2020/039736

§ 371 Date: Dec. 27, 2021

Related U.S. Application Data
(60) Provisional application No. 62/867,184, filed on Jun. 26, 2019.

(51) Int. Cl. C12Q 1/00; C12Q 1/24; C12Q 1/66

(52) U.S. Cl. CPC C12Q 1/008; C12Q 1/24; C12Q 1/66

(57) ABSTRACT

The present invention relates methods for rapidly detecting the presence of bacteria in biological solutions regardless of their origin in non-laboratory environments. The present invention further relates to methods for binding, capturing, and concentrating the bacteria in a given sample.

### CROSS-REFERENCE TO RELATED APPLICATIONS

The present application claims priority of U.S. Provisional Patent Application No. 62/867,184 filed Jun. 26, 2019. The entire contents of which are hereby incorporated by reference.

### FIELD OF THE INVENTION

The present invention relates to several methods to detect gram positive and gram negative pathogens in various biological fluids and surfaces. For the purposes of the present invention, all references as cited herein are incorporated by reference in their entireties.

### BACKGROUND OF THE INVENTION

The present invention relates methods for rapidly detecting the presence of bacteria in biological solutions regardless of their origin in non-laboratory environments. The present invention further relates to methods for binding, capturing, and concentrating the bacteria in a given sample. Particular attention is given to the ability to determine gram type of said bacteria, particularly gram positive organisms.

Gram positive organisms are universally found in samples such as water, blood, urine, food stuffs, and many others collected from humans, animals and plants. A whole series of gram positive pathogenic bacteria such as: Staphylococcus, Enterococcus, Streptococcus, Listeria, Bacillus, Clostridium and mycobacteria are particular important in human and animal health, as well as the food industry. The rapid detection of these, and many other bacteria are important in the diagnosis and treatment of many infectious diseases.

Staphylococcus, Streptococcus, and Enterococcus are rapidly developing antibiotic resistance and they have become among the most difficult pathogens to treat. They are associated with infectious diseases such as: VRE-(vancomycin resistant Enterococcus) and MRSA-(multi-resistant Staph aureus). This dilemma has led to numerous investigations into the nature of antibiotic resistance, and procedures for eliminating the problem by limiting the overuse of antibiotics in humans and animals.

Samples containing these pathogenic bacteria are often present in low numbers (10¹-10³ organisms/mL) and in matrixes of interfering substance. Techniques are therefore often needed to enhance their numbers, and to remove or block the inhibitory effect of interfering substances.

The present patent will enumerate a number of different approach that allow the detection of low numbers of bacterial cells, as well ameliorate the problems presented by the sample's matrix. This patent further describes techniques that address the secondary problem of identifying the gram status of the bacteria of interest.

In most methods used to detect low numbers of organisms an initial enrichment of the bacteria present requires various culture technologies. These culturing techniques normally require from 18 to 72 hours to complete. For example, the standard enrichment times for Staphylococcus aureus is between 16 to >48 hours (Kim J, Gregson D B, Ross T, Laupland K B, J Infect. 2010 September; 61(3):197-204) and for Listeria are reported to be between 4-7 days (ISO 11290-1). If Gram status determinations are required as well, then differential bacterial plate culturing [under sterile conditions] require an additional 24-36 hours for reliable results. In many situations, such as human and animal health, food stuffs preparation, and cleanliness determinations long detection time could be detrimental in a critical analysis. In this patent a number of different approaches are suggested to eliminate the need for pre-culturing of the biological sample prior to detection of the pathogenic organisms. Among these are filtration technologies and binding technologies that capture and concentrate the organism present in the biological sample prior to testing.

It is clear that the requirements for detection of low numbers (10¹-10³ organisms/mL) of pathogenic bacteria in non-laboratory environments must have the following characteristics: high sensitivity, high specificity, gram status, rapid, ease of use, inexpensive and reliable. The above requirements are solved by the subject matter detailed in the patent claims. The following figures are useful in illustrating the invention.

### BRIEF DESCRIPTION OF THE DRAWINGS

The following drawings schematically show preferred embodiments of the apparatus for performing the methods of the inventions.

FIG. 1 is schematic of the disposable lateral/vertical flow through cassette of first embodiment of the present inventions designed to determine bacterial concentration in a biological solution in which the bacteria have not been removed from the initial solution.

FIG. 2 is a perspective of a cross-section along the longitudinal axis of the disposable lateral/vertical flow through cassette (FIG. 1) of the present inventions.

FIG. 3 is a schematic of disposable lateral/vertical flow through device (FIG. 1) before and after insertion into a luminometer.

FIG. 4 is a perspective of a cross-section along the longitudinal axis of a disposable vertical flow through cassette designed to determine bacterial concentration in a biological solution in which the bacteria have not been removed from the initial solution for a second embodiment of the present inventions.

FIG. 5 is a schematic of disposable vertical flow through device (FIG. 4) after insertion into a luminometer.

FIG. 6 is a schematic of a “Paddlewheel” collection and concentration device for a third embodiment of present inventions in which bacteria will be captured directly from a biological solution.

FIG. 7 is a schematic of a “Paddlewheel” collection and concentration device (FIG. 6) inserted into a sample collection container to remove bacteria from a biological solution.

FIG. 8 is a perspective of a cross-section along the longitudinal axis of a disposable lateral/vertical flow through cassette to be used in conjunction with (FIG. 6) for the third embodiment of present inventions in which bacterial will be capture from a biological solution.

FIG. 9 is a perspective of a cross-section along the longitudinal axis of a disposable vertical flow through cassette designed to determine bacterial concentration in a biological solution in which the bacteria have been removed from the initial solution for a fourth embodiment of the present inventions.

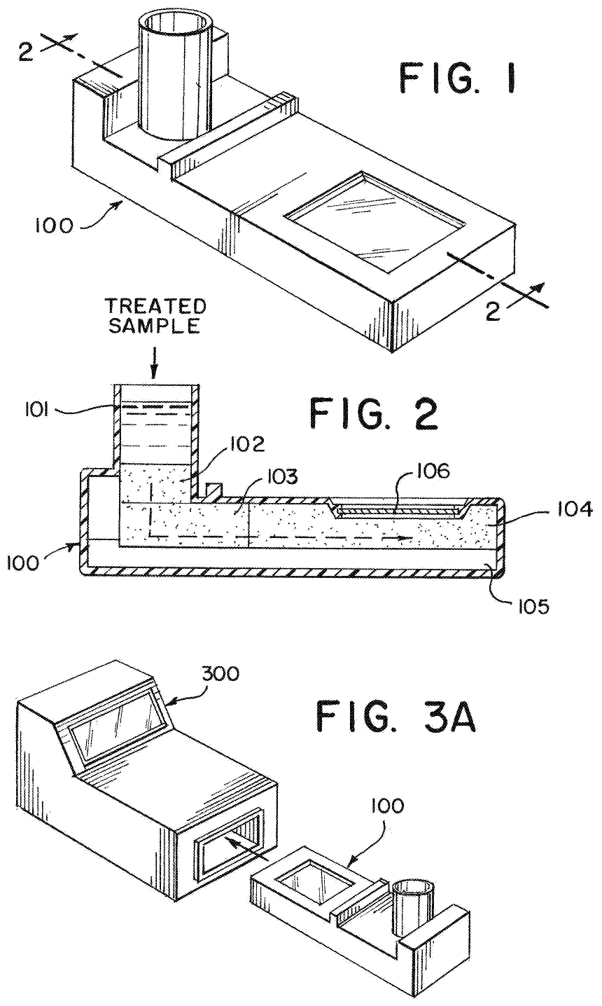

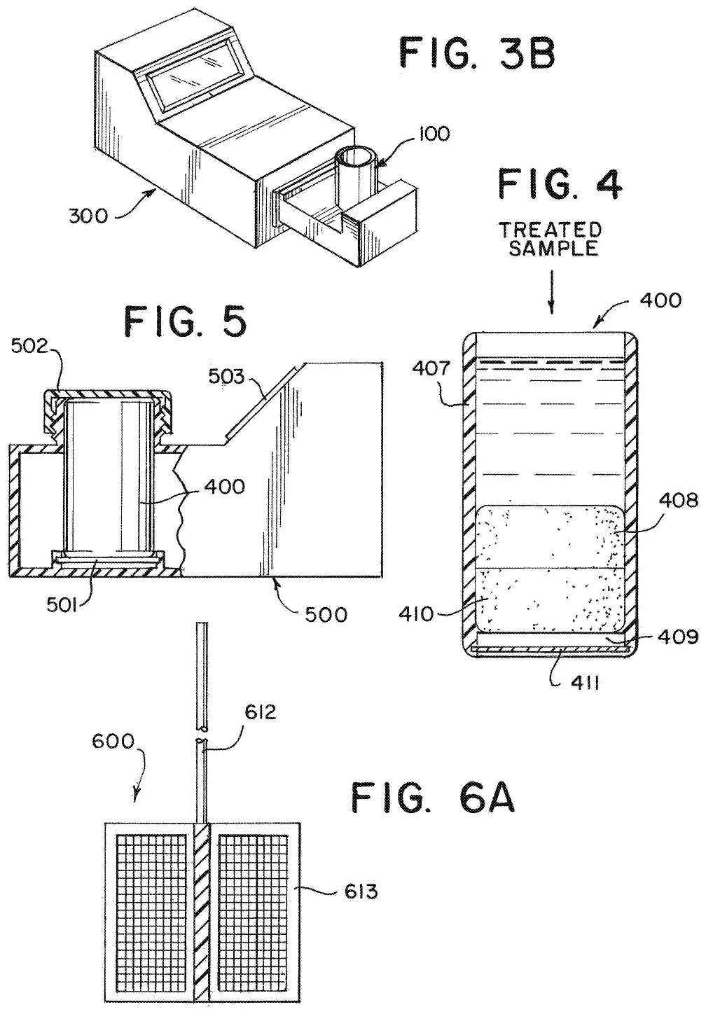

### SUMMARY OF THE INVENTION

The present invention features methods and kits for detecting gram positive and gram negative bacteria.

In one embodiment of the invention there is provided a method for determining amounts of bacteria in a liquid sample comprising the steps of:
i) Filtration of the liquid sample to produce a filtrate
ii) Treatment of the filtrate with a non-ionic surfactant which lyses non-microbial cells (somatic cells) to produce a first solution
iii) Treatment of the first solution with an ATP eliminating enzyme to produce a second solution
iv) Treatment of the second solution with an ATP eliminating enzyme inhibitor to give a third solution
v) Treatment of the third solution with a microbial lysing agent to give a fourth solution
vi) Treatment of the fourth solution with a Luciferin/Luciferase reagent to give a fifth solution and
vii) Quantitation of bacteria in the fifth solution by luminescence; with the proviso that the liquid sample is not bovine milk.

Preferably the liquid sample is obtained from a surface using a cloth, gauze, swab, wipe, non-woven fiber or sponge or collected in a container.

Preferably the liquid sample is a biological liquid sample.

Preferably the biological liquid sample is chosen from the group consisting of blood, plasma, serum, lactation products, amniotic fluids, sputum, saliva, urine, semen, cerebrospinal fluid, uterine fluids, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

Preferably the liquid sample is a fluid or specimen obtained from an environmental source selected from a fluid or specimen obtained or derived from food products, food produce, poultry, meat, fish, beverages, dairy product, eggs, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, animal colony facilities, or any combinations thereof.

Preferably the liquid sample is urine. Preferably the liquid sample is human urine. Preferably the non-ionic surfactant is chosen from the group consisting of Neonol AF9-10 (Nonoxynol-9), saponin, amphipathic glycosides Triton X-100 and Lubrol.

Preferably the non-ionic surfactant is Neonol AF9-10.

Preferably the ATP eliminating enzyme comprises at least one member selected from the group consisting of apyrase, ATPase, alkaline phosphatase, acidic phosphatase, hexokinase, adenosine triphosphatase, adenosine phosphate deaminase and luciferase.

Preferably the ATP eliminating enzyme is Apyrase.

Preferably the ATP eliminating enzyme inhibitor is an ionic surfactant.

Preferably the ionic surfactant is selected from the group consisting of anionic surfactants cationic surfactants and zwitterionic surfactants.

Preferably the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

Preferably the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

Preferably the ionic surfactant is selected from DTAB, CTAB and BDDABr.

Preferably the zwitterionic surfactant is chosen from the group consisting of sulfobetaine-3-10.

Preferably the ATP eliminating enzyme inhibitor is selected from the group consisting of vanadates and hydroxy apatites and their derivatives.

Preferably the bacterial releasing agent is a bacteriophage lytic enzyme (endolysin) or modified lytic enzyme (genetic or chimeric), quaternary amines, ionic and or non-ionic surfactants.

Preferably the bacteriophage lytic endolysin is selected from Lysostaphin, LysK, Lyse5h, LambdaSa2, OSH3b, and KSN383, LysA, LysA2, LysgaY, truncated lambda Sa2, H5CHAP-Lyso, Lysolv123-H5CHAP-OSH3b, and PlyC.

Preferably the Luciferin/Luciferase reagent is chosen from the group consisting of Hygiena ATP Biomass Kit #CCK4, Promega Bright Glo system and any formulations which contain naturally occurring or genetically recombinant Luciferase.

Preferably the quantization of bacteria is performed on a liquid or solid state substrate.

Preferably the quantization of bacteria is-performed on a solid-state substrate.

Preferably the solid-state substrate is selected from polyvinyl alcohol, Porex membranes, Whatman, paper membranes, Ahlstrom membranes, Nitrocellulose membranes, and Whatman Nytran membranes and Nylon membranes.

Preferably the bacteria is gram positive or gram negative bacteria.

Preferably the gram-positive bacteria are elected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

Preferably the gram-negative bacteria are selected from the group consisting of Escherichia coli, Klebsiella pneumoniae, Pseudomonas aeruginosa and Proteus mirabilis.

Preferably the bacteria are selected from the group consisting of Streptococcus agalactiae, Streptococcus dysgalactiae, Streptococcus spp., Staphylococcus aureus, Staphylococcus spp., Escherichia coli, Klebsiella pneumoniae, Pseudomonas aeruginosa, Enterococcus spp., and Proteus mirabilis.

In another embodiment of the invention there is provided a method for determining amounts of bacteria in a liquid sample comprising the steps of:
i) Filtration of the liquid sample through a filter whose porosity is such that bacteria and somatic cells remains resident on the filter to give a residue on the filter;
ii) Treatment of the retained residue with a non-ionic surfactant which lyses non-microbial cells (somatic cells) selectively, thereby removing their ATP from the residue on the filter by application of positive pressure.
iii) Treatment of the retained microbial residue with a bacterial releasing agent capable of lysing bacterial cells to give a released ATP second residue;
iv) Treatment of the released ATP second residue with a Luciferin/Luciferase reagent to give a third solution and
v) Quantitation of bacteria in the third solution by luminescence; with the proviso that the liquid sample is not bovine milk.

Preferably the liquid sample is obtained from a surface using a cloth, gauze, swab, wipe, non-woven fiber or sponge or collection container.

Preferably the liquid sample is a biological liquid sample.

Preferably the biological liquid sample is chosen from the group consisting of blood, plasma, serum, uterine fluids, lactation products, amniotic fluids, sputum, saliva, urine, semen, cerebrospinal fluid, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

Preferably the liquid sample is a fluid or specimen obtained from an environmental source selected from a fluid or specimen obtained or derived from food products, food produce, poultry, eggs, meat, fish, beverages, dairy product, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, animal colony facilities, or any combinations thereof.

Preferably the liquid sample is urine.

Preferably the liquid sample is human urine.

Preferably the non-ionic surfactant is chosen from the group consisting of Neonol AF9-10 (Nonoxynol-9), saponin, amphipathic glycosides Triton X-100 and Lubrol.

Preferably the bacterial releasing agent is a bacteriophage lytic enzyme (endolysin) or modified lytic enzyme (genetic or chimeric), quaternary amines, ionic and or non-ionic surfactants.

Preferably the ionic surfactant is selected from the group consisting of anionic surfactants cationic surfactants and zwitterionic surfactants.

Preferably the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

Preferably the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

Preferably the ionic surfactant is selected from DTAB, CTAB and BDDABr.

Preferably the zwitterionic surfactant is chosen from the group consisting of sulfobetaine-3-10.

Preferably the bacteriophage lytic endolysin is selected from Lysostaphin, LysK, LyseSh, LambdaSa2, OSH3b, and KSN383, LysA, LysA2, LysgaY, truncated lambda Sa2, H5CHAP_Lyso, LysoM23-H5CHAP-OSH3b, and PlyC.

Preferably the Luciferin/Luciferase reagent is chosen from the group consisting of Hygiena ATP Biomass Kit #CCK4, Promega Bright Glo system and any formulations which contain naturally occurring or genetically recombinant Luciferase.

Preferably the bacteria are gram positive or gram negative bacteria.

Preferably the gram-positive bacteria are selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

Preferably the gram-negative bacteria are selected from the group consisting of Escherichia coli, Klebsiella pneumoniae, Pseudomonas aeruginosa, and Proteus mirabilis.

Preferably the bacteria are selected from the group consisting of Streptococcus agalactiae, Streptococcus dysgalactiae, Streptococcus spp., Staphylococcus aureus, -aH-d Staphylococcus spp., Escherichia coli, Klebsiella pneumoniae, Pseudomonas aeruginosa, Enterococcus spp., and Proteus mirabilis.

In another embodiment of the invention there is provided a method for determining amounts of bacteria in a liquid sample comprising the steps of:
i) Contacting the liquid sample with a material which selectively attracts bacteria
ii) Separating the bacteria from the material and placing the bacteria into solution
iii) Treatment of the bacterial solution with a bacterial releasing agent capable of lysing bacterial cells to give a released ATP second solution
iv) Treatment of the second solution with a Luciferin/Luciferase reagent to give a third solution and
v) Quantitation of bacteria in the third solution by luminescence.

Preferably the material is selected from the group consisting of antibody coated surfaces, lectin coated surfaces, lytic enzyme binding domains coated surfaces, glass wool membranes and treated glass surfaces.

Preferably the material is on a dipstick or paddlewheel. Preferably the liquid sample is a biological liquid sample.

Preferably the biological liquid sample is chosen from the group consisting of blood, plasma, serum, uterine fluids, lactation products, amniotic fluids, sputum, saliva, urine, semen, cerebrospinal fluid, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

Preferably the liquid sample is a fluid or specimen obtained from an environmental source selected from a fluid or specimen obtained or derived from food products, food produce, poultry, eggs, meat, fish, beverages, dairy product, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, animal colony facilities, or any combinations thereof.

Preferably the liquid sample is milk.

Preferably the milk is bovine milk. Preferably the liquid sample is urine. Preferably the liquid sample is human urine.

Preferably the liquid sample is obtained from a surface using a cloth, gauze, swab, wipe, non-woven fiber or sponge or container.

Preferably the bacterial releasing agent is a bacteriophage lytic enzyme (endolysin) or modified lytic enzyme (genetic or chimeric), quaternary amines, ionic and or non-ionic surfactants.

Preferably the ionic surfactant is selected from the group consisting of anionic surfactants cationic surfactants and zwitterionic surfactants.

Preferably the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

Preferably the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

Preferably the ionic surfactant is selected from DTAB, CTAB and BDDABr.

Preferably the zwitterionic surfactant is chosen from the group consisting of sulfobetaine-3-10.

Preferably the bacteriophage lytic endolysin is selected from Lysostaphin, LysK, Lyse5h, LambdaSa2, OSH3b, and KSN383, LysA, LysA2, LysgaY, truncated lambda Sa2, HSCHAP_Lyso, LysoM23-H5CHAP-OSH3b, and PlyC.

Preferably the Luciferin/Luciferase reagent is chosen from the group consisting of Hygiena ATP Biomass Kit #CCK4, Promega Bright Glo system and any formulations which contain naturally occurring or genetically recombinant Luciferase.

Preferably the bacteria are gram positive or gram negative bacteria.

Preferably the gram-positive bacteria are selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

Preferably the gram-negative bacteria are selected from the group consisting of Escherichia coli, Klebsiella pneumoniae, Pseudomonas aeruginosa and Proteus mirabilis.

Preferably the bacteria is selected from the group consisting of Streptococcus agalactiae, Streptococcus dysgalactiae, Streptococcus spp., Staphylococcus aureus, and Staphylococcus spp., Escherichia coli, Klebsiella pneumoniae, Pseudomonas aeruginosa, Enterococcus spp., and Proteus mirabilis.

In another embodiment of the invention there is provided method for determining amounts of bacteria in a liquid sample comprising the steps of:
i) Placing the liquid sample in a first container containing a non-ionic surfactant which lyses non-microbial (somatic cells) and an ATP eliminating enzyme to produce a first solution;
ii) Transferring the first solution to a second container containing an ATP eliminating enzyme inhibitor, a microbial lysing agent and a Luciferin/Luciferase reagent to give a second solution and
iii) Quantitation of bacteria in the second solution by luminescence.

Preferably the liquid sample is obtained from a surface using a cloth, gauze, swab, wipe, non-woven fiber or sponge.

Preferably the liquid sample is a biological liquid sample.

Preferably the biological liquid sample is chosen from the group consisting of blood, plasma, serum, lactation products, amniotic fluids, sputum, saliva, urine, semen, cerebrospinal fluid, uterine fluids, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

Preferably the liquid sample is a fluid or specimen obtained from an environmental source selected from a fluid or specimen obtained or derived from food products, food produce, poultry, meat, fish, beverages, dairy product, eggs, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, animal colony facilities, or any combinations thereof.

Preferably the liquid sample is urine. Preferably the liquid sample is human urine.

Preferably the non-ionic surfactant is chosen from the group consisting of Neonol AF9-10 (Nonoxynol-9), saponin, amphipathic glycosides Triton X-100 and Lubrol.

Preferably the non-ionic surfactant is Neonol AF9-10.

Preferably the ATP eliminating enzyme comprises at least one member selected from the group consisting of apyrase, alkaline phosphatase, acidic phosphatase, hexokinase, adenosine triphosphatase, adenosine phosphate deaminase and luciferase.

Preferably the ATP eliminating enzyme is Apyrase.

Preferably the ATP eliminating enzyme inhibitor is an ionic surfactant.

Preferably the ionic surfactant is selected from the group consisting of anionic surfactants cationic surfactants and zwitterionic surfactants.

Preferably the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

Preferably the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

Preferably the ionic surfactant is selected from DTAB, CTAB and BDDABr.

Preferably the zwitterionic surfactant is chosen from the group consisting of sulfobetaine-3-10.

The method according to claim 81 wherein the ATP eliminating enzyme inhibitor is selected from the group consisting of vanadates and hydroxyapatites and their derivatives.

The method according to claim 70 wherein the microbial lysing agent is a bacteriophage lytic enzyme (endolysin) or modified lytic enzyme (genetic or chimeric).

Preferably the bacteriophage lytic endolysin is selected from Lysostaphin, LysK, Lyse5h, LambdaSa2, OSH3b, and KSN383, LysA, LysA2, LysgaY, truncated lambda Sa2, HSCHAP_Lyso, LysoM23-H5CHAP-OSH3b, and PlyC.

Preferably the Luciferin/Luciferase reagent is chosen from the group consisting of Hygiena ATP Biomass Kit #CCK4, Promega Bright Glo system and any formulations which contain naturally occurring or genetically recombinant Luciferase.

Preferably the bacteria is gram positive or gram negative bacteria.

Preferably the gram-positive bacteria are selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

Preferably the gram-negative bacteria are selected from the group consisting of Escherichia coli, Klebsiella pneumoniae, Pseudomonas aeruginosa, and Proteus mirabilis.

Preferably the gram-positive bacteria are selected from the group consisting of Streptococcus agalactiae, Streptococcus dysgalactiae, Streptococcus spp., Staphylococcus aureus, Staphylococcus epidermidis, and Staphylococcus spp.

Preferably the bacteria is selected from the group consisting of Streptococcus agalactiae, Streptococcus dysgalactiae, Streptococcus spp., Staphylococcus aureus, -ami Staphylococcus spp., Escherichia coli, Klebsiella pneumoniae, Pseudomonas aeruginosa, Enterococcus spp., and Proteus mirabilis.

In another embodiment of the invention there is provided method for determining amounts of bacteria in a liquid sample comprising the steps of:
i) Placing the liquid sample in a first container containing a non-ionic surfactant which lyses non-microbial (somatic cells) and an ATP eliminating enzyme to produce a first solution;
ii) Contacting the first solution with a pad impregnated with an ATP eliminating enzyme inhibitor, which is in contact with a second pad impregnated with a microbial lysing agent which is in contact with a third pad impregnated with a Luciferin/Luciferase;
iii) Quantitation of bacteria on the third pad by luminescence.

Preferably the liquid sample is obtained from a surface using a cloth, gauze, swab, wipe, non-woven fiber or sponge or collected in a container.

Preferably the liquid sample is a biological liquid sample.

Preferably the biological liquid sample is chosen from the group consisting of blood, plasma, serum, lactation products, amniotic fluids, sputum, saliva, urine, semen, cerebrospinal fluid, uterine fluids, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

Preferably the liquid sample is a fluid or specimen obtained from an environmental source selected from a fluid or specimen obtained or derived from food products, food produce, poultry, meat, fish, beverages, dairy product, eggs, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, animal colony facilities, or any combinations thereof.

Preferably the liquid sample is urine. Preferably the liquid sample is human urine.

Preferably the non-ionic surfactant is chosen from the group consisting of Neonol AF9-10 (Nonoxynol-9), saponin, amphipathic glycosides Triton X-100 and Lubrol.

Preferably the non-ionic surfactant is Neonol AF9-10.

Preferably the ATP eliminating enzyme comprises at least one member selected from the group consisting of apyrase, alkaline phosphatase, acidic phosphatase, hexokinase, adenosine triphosphatase, adenosine phosphate deaminase and luciferase.

Preferably the ATP eliminating enzyme is Apyrase.

Preferably the ATP eliminating enzyme inhibitor is an ionic surfactant.

Preferably the ionic surfactant is selected from the group consisting of anionic surfactants cationic surfactants and zwitterionic surfactants.

Preferably the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

Preferably the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

Preferably the ionic surfactant is selected from DTAB, CTAB and BDDABr.

Preferably the zwitterionic surfactant is chosen from the group consisting of sulfobetaine-3-10.

Preferably the ATP eliminating enzyme inhibitor is selected from the group consisting of vanadates and hydroxy apatites and their derivatives.

Preferably the microbial lysing agent is a bacteriophage lytic enzyme (endolysin) or modified lytic enzyme (genetic or chimeric).

Preferably the bacteriophage lytic endolysin is selected from Lysostaphin, LysK, LyseSh, LambdaSa2, OSH3b, and KSN383, LysA, LysA2, LysgaY, truncated lambda Sa2, H5CHAP_Lyso, LysoM23-H5CHAP-OSH3b, and PlyC.

Preferably the Luciferin/Luciferase reagent is chosen from the group consisting of Hygiena ATP Biomass Kit #CCK4, Promega Bright Glo system and any formulations which contain naturally occurring or genetically recombinant Luciferase.

Preferably the bacteria is gram positive or gram negative bacteria.

Preferably the gram-positive bacteria are selected from Staphylococcus spp.,

Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

Preferably the gram-negative bacteria are selected from the group consisting of Escherichia coli, Klebsiella pneumoniae, Pseudomonas aeruginosa, and Proteus mirabilis.

Preferably the gram-positive bacteria are selected from the group consisting of Streptococcus agalactiae, Streptococcus dysgalactiae, Streptococcus spp., Staphylococcus aureus, Staphylococcus epidermidis, and Staphylococcus spp.

Preferably the bacteria is selected from the group consisting of Streptococcus agalactiae, Streptococcus dysgalactiae, Streptococcus spp., Staphylococcus aureus, Staphylococcus spp., Escherichia coli, Klebsiella pneumoniae, Pseudomonas aeruginosa, Enterococcus spp., and Proteus mirabilis

In another embodiment of the invention there is provided a device as depicted in FIG. 4.

In another embodiment of the invention there is provided a device as depicted in FIG. 5.

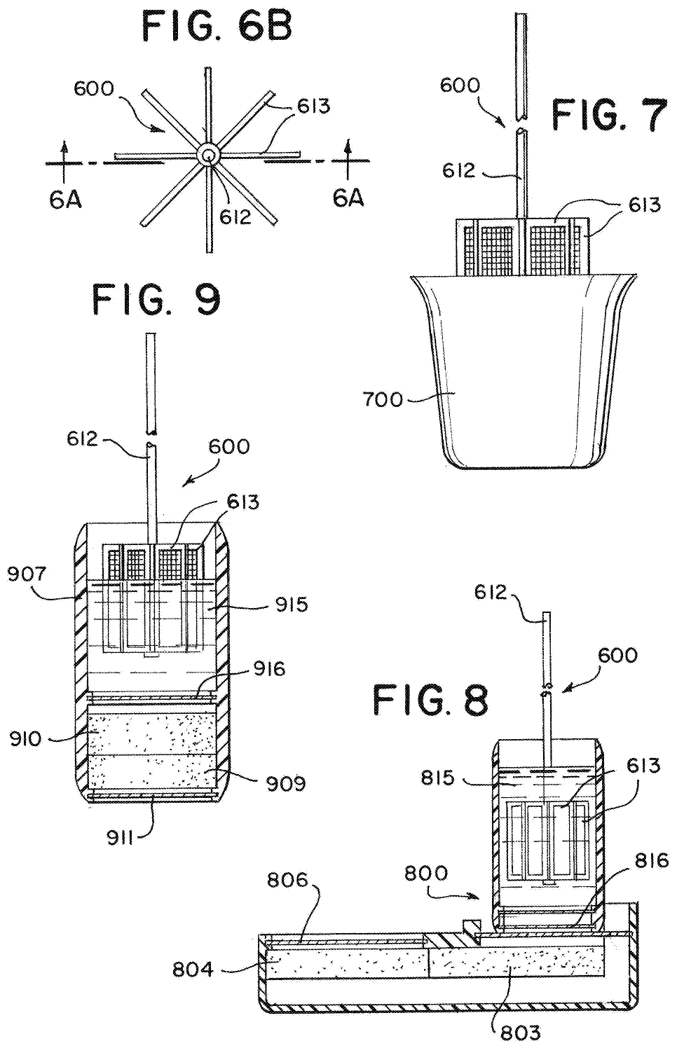

In another embodiment of the invention there is provided a device as depicted in FIG. 6.

In another embodiment of the invention there is provided device as depicted in FIG. 8.

In another embodiment of the invention there is provided device as depicted in FIG. 9.

### DETAILED DESCRIPTION OF THE INVENTION

### Examples

The following examples are intended to illustrate the present invention without limitations.

### Example 1

Determination of Optimum Method to Filter Sample.

In many assays, it may be necessary to pretreat the sample to remove interfering substances or other endogenous materials that may be present. In one procedure, milk (with endogenous bacteria) was filtered via gravity flow for 2 min using 2 different commercially available filter papers (after evaluation of numerous filter media). The filter media of choice are: Ahlstrom 222 (A222) and Ahlstrom 142 (A142). The filter papers where supplied by Ahlstrom Corporation. Samples (100 uL) prior to and after filtering on Ahlstrom 222 (A222) and Ahlstrom 142 (A142)) were tested for ATP with 50 uL of Promega luciferin-luciferase reagent (containing 25 mM HEPES buffer (pH 7.5), 40 pg luciferase, 100 pM luciferin, and 10 mM MgSO4), as well as for colony forming units (CFUs) via serial dilution on rich media tryptic soy agar (TSA) plates. The endogenous ATP appears to have largely (70%) bound to the A222 filter, while nearly 95% of the bacteria appear to have passed through this filter. The A142 filter does not appear useful in this process since it does not allow the bacteria to pass through. There was no testing for somatic cells in the filtrate of the A222 filter, such that the reduction in ATP after filtration might reflect the capturing of the somatic cells on the filter and the resultant loss of intracellular somatic cells stores of ATP. All determinations of ATP concentration were performed using a Hygiena Ensure luminometer. In a second procedure, urine samples (with endogenous bacteria) were filtered to remove solid particles and other endogenous materials via gravity flow for >1 minute using 2 different commercially available filter papers (after evaluation of numerous filter media). The filter media of choice was: Ahlstrom 222 (A222). The filter papers where supplied by Ahlstrom Corporation. Samples (100 uL) prior to and after filtering on Ahlstrom 222 (A222) were tested for ATP with 50 uL of Promega luciferin-luciferase reagent (containing 25 mNI HEPES buffer (pH 7.5), 40 pg luciferase, 100 pM luciferin, and 10 mM MgSO4), as well as for colony forming units (CFUs) via serial dilution on rich media tryptic soy agar (TSA) plates. Some (<10%) of the endogenous ATP appears to bound to the A222 filter, while nearly 95% of the bacteria appear to have passed through the filter. There was no testing for somatic cells in the filtrate of the A222 filter, such that the reduction in ATP after filtration might reflect the capturing of the somatic cells on the filter and the resultant loss of intracellular somatic cells stores of ATP. All determinations of ATP concentration were performed using a Hygiena Ensure luminometer.

### Example 2

Determination of Usefulness of Luciferin/Luciferase Reagent in for ATP Concentrations in a Sample

To evaluate the usefulness of Luciferin/Luciferase Reagent in measuring ATP concentrations in various samples we evaluate samples of urine (human) and milk. Some biological fluids are known to have endogenous ATP which potentially could interfere with a bioluminescent assay and we determined the levels of some endogenous ATP. In the first procedure, the biological sample was commercially available milk samples which were prepared containing various amounts of ATP standard from Sigma Chemical (#A2383.) to produce milk samples with concentrations of 10⁻⁶M to 10⁻¹⁰ M. In each test, 50 uL of Promega luciferin-luciferase reagent (containing 25 mM HEPES buffer (pH 7.5), 40 pg luciferase, 100 pM luciferin, and 10 mM MgSO4). were added to milk sample and light output was determine using Hygiena Ensure System. All measurements were performed by first obtaining signal output of untreated milk sample devoid of additional ATP (blank) and final ATP readings for each sample described above were corrected for the blank. In the second procedure, the biological sample were urine (human) samples which were prepared containing various amounts of ATP standard obtained from Sigma Chemical (#A2383.) to produce milk samples with concentrations of 10⁻⁶M to 10⁻¹⁰M ATP. These samples were tested as described above. In all cases the ATP levels were detected as expected for the ATP concentrations noted above. For comparison, standard solutions of ATP prepared in buffer were tested against their blank, and results similar to the milk and urine study were obtained. This indicates that neither milk samples nor urine appear to hinder the Luciferin-Luciferase reaction, and are useful for the determination of bacteria in many biological samples.

### Example 3

Determination of Methods to Rupture Somatic Cells Present in Biological Samples.

In the determination of ATP levels in biological sample it is often necessary to eliminate somatic cells present as that may rupture and release ATP during experimentation, which would interfere with the measurement of ATP from bacterial sources in a future step. The removal of interference from somatic cell ATP can be accomplished by filtration of such cells, which if often time consuming or by lysing (rupturing) the somatic cells (prior to the lysing of the bacterial cells) and then eliminating the ATP so produced.

A number of surfactants (detergents) were evaluated for their ability to rupture the somatic cells present in raw biological sample samples. Among the reagents tested were Triton X 100 (Sigma Chemical) and Neonol AF9-I0 (Nonoxynol-9) (Elarum Petrochemicals). Determinations using both surfactants were performed on milk and urine (human) samples, in which somatic cell counts were predetermined. It was determined that the Neonol-9-I0 was superior to the Triton X100 in its ability to rupture somatic cells in under 90 seconds. The number of somatic cell ruptured was determined by quantifying the ATP released in 50 uL samples of both milk and urine (as a function of time) using 50 uL of Promega luciferin-luciferase reagent (containing 25 mM HEPES buffer (pH 7.5), 40 pg luciferase, 100 pM luciferin, and 10 mM MgSO4). Bioluminescent measurements were performed using a Hygiena Ensure luminometer and all readings were blank corrected.

### Example 4

Determination of Method to Eliminate Endogenous ATP Present in Biological Sample.

To demonstrating the ability to eliminate the endogenous ATP from biological samples (from both somatic cells and sample matrix), we selected Apyrase, an ATPase family enzyme, from Sigma Chemical (A6535, ATPase 2:200 units/mg protein). All assays were performed using 50 uL of milk or urine (human) and 50 uL of Promega luciferin-luciferase reagent (containing 25 mM HEPES buffer (pH 7.5), 40 pg luciferase, 100 pM luciferin, and 10 mM MgSO4). Bioluminescent measurements were determined using the Hygiena Ensure System. The test was performed in both 100% and 50% milk and urine (human). Results indicate that Apyrase works well in both 100% and 50% biological samples. At a concentration of 284 mUnits the apyrase is able to deplete the endogenous ATP in 50 μL of milk or urine (human) in less than 30 seconds. The diminution of ATP was so fast with higher concentrations of Apyrase resulting in the inability to take meaningful measurements, since all of the ATP was gone in <10 seconds. There are numerous commercially available ATP-degrading enzymes that can be tested for this purpose, but in our system, the Apyrase enzyme appears more than sufficient.

### Example 5

Determination of “Eliminating Reagents” for Excess Apyrase Enzyme Present in Biological Samples after Treatment to Eliminate Endogenous ATP (Example #4)

It is important to eliminate any excess Apyrase that may remain in the biological sample after treatment with the Apyrase enzyme that was used to eliminate endogenous ATP in Example #4. We examined a number of anionic and cationic surfactants (detergents) to determine those that are most effective at inactivate Apyrase. Among the reagents evaluated were: dimethyldioctadecylammonium chloride, laureltrimethylammonium bromide (DTAB), benzyldimethyldodecylammonium bromide (BDDABr), and cetyl trimethylammonium bromide (CTAB). The experiment was performed by adding between 0.02% and 1% of the selected surfactant to 50 ul of milk or urine (human) that had been treated previously with 284 mU of Apyrase as detailed in example #4. The levels of ATP were confirmed by adding 50 uL of Promega luciferin-luciferase reagent (containing 25 mM HEPES buffer (pH 7.5), 40 pg luciferase, 100 pM luciferin, and 10 mM MgSO4). As expected the readings determined on the Hygiena Ensure Luminometer were too low to measure, since all of the ATP had already been eliminated by Apyrase treatment in Example #4. After the initially readings were determined as described above, 10 uL of a 10⁻⁸M solution of ATP standard (Sigma Chemical) was added to the samples above and the bioluminescent signal was determined on a Hygiena Ensure Luminometer. As expected a signal was now detected, since all of the excess Apyrase added earlier had been eliminated by the surfactants being evaluated. The best results were seen when the detergents benzyldimethyldodecylammonium bromide (BDDABr) at 0.05% concentration and, laureltrimethylammonium bromide (DTAB) at 0.5% and 1.0% concentrations were added to the samples.

### Example 6

Determination of the Ability of Both Streptococcal and Staphylococcal Phage Lytic Enzyme (Endolysins or Peptidoglycan Hydrolases) to Effectively Rupture Gram Positive Bacteria in Biological Samples.

One of the enumerated goals of this invention is to target and detect specifically gram positive organisms such as: S. aureus, Coagulase negative staphylococci (CoNS) and S. uberi etc. Therefore, it is important to identify phage lytic enzymes that can effectively rupture the cell walls of the aforementioned pathogens in the presence of biological sample such as milk and urine. Lysostaphin (Lyso) and Streptococcal phage endolysin (PlyC), as well many other phage lytic enzymes developed in our laboratories have shown high activity against the major Gram positive pathogens in many biological fluids. The action against specific gram-positive organisms of these enzymes has been verified in PBS buffered solutions, but their ability to rupture bacteria had to be verified to in biological sample such as milk and urine. The first candidates tested in for their activity in milk and urine were Lysostaphin (Lyso) which attacks S. aureus, and Streptococcal phage lytic enzyme (PlyC) which attacks Strep uberis, Strep A and Strep C. In all reactions, a concentration a of 0.05% of the phage lytic enzyme was used to evaluate the time required to rupture the cell wall of gram-positive bacteria in milk, as well as in a Phosphate-buffered saline (PBS) solution. It was determined that that in one (1) to three (3) minutes, PlyC can rupture up to 6 logs of S. uberis, S. c and S. a in PBS buffer, as well as milk or urine (human). The degree of lysing was determined by bioluminescence produced by the ATP released from the ruptured cells as described earlier. We then lowered the bacterial load to 2 logs of S. uberis in PBS buffer and in either milk, or urine (human). It was determined that the PlyC can eradicate this bacterial load in 2 minutes or less. Similar experiments were performed with Lysostaphin (Lyso), which attacks S. aureus, and we determined we that can eradicate up to 6 logs of S. aureus in PBS buffer in under 3 minutes. We then lowered the bacterial load to 2 logs of S. aureus in PBS buffer and either milk or urine (human), and it was determined that the Lyso can eradicate this bacterial load in 2 minutes or less.

### Example 7

Filtration of a Liquid Biological Sample Through a Filter Whose Porosity is Such that Bacteria Remains Resident on the Filter to Give a Residue on Such Filter

In some assays, it may be necessary to pre-treat the liquid biological sample to remove interfering substances, such as somatic cells and endogenous ATP which may interfere with subsequent determination of number of bacterial cells present. In one procedure, a sample of urine (human) was pretreated by filtering through a 0.45-micron filter disk (MicronSep Nitrocellulose membrane filters (4641K36), (Thomas Scientific Company) which is bond to a sample vessel by a suitable solvent (MEK). The biological sample (100 uL) is forced through the filter disk by the application of positive pressure provided by a luer-lok syringe or similar device. The filter disk allows all small molecule interfering substance to pass through the filter, but retains all the bacterial and somatic cells present in the original biological sample on the surface of the filter. The bacterial and somatic cells remaining on the surface of the filter membrane were then washed twice, under positive pressure, with 50 uL of 0.05% Nonoxynol-9 solution (Neonol AF9-10, Elarum Corporation), a non-ionic surfactant which selectively lyses non-microbial cells (somatic cells), thereby removing them and their ATP from the residue remaining on the filter. The bacterial cells which remain on the surface of the filter disk are then treated with a 50 uL of Bacterial Releasing Agent, (BRA10X, New Horizon Diagnostics, Inc., Baltimore, Md.) and 50 uL of Promega luciferin-luciferase reagent (containing 25 mM HEPES buffer (pH 7.5), 40 pg luciferase, 100 pM luciferin, and 10 mM MgSO4). The resultant bioluminescent signal produced by the ATP released by the captured bacterial cells is read using a Hygiena Ensure luminometer and all readings were blank corrected.

### Example 8

Determination of the Number Bacteria in a Liquid Biological Sample with a Material which Selectively Attracts Bacteria and Allows their Removal from the Sample.

In order to enumerate the low levels of bacteria that are often required in an analysis biological liquid samples, it may be advantageous to concentrate the bacteria in the liquid sample by extracting them for a large volume of sample without the need for difficult and time consuming filtration steps. In one procedure, a 2 mL of milk was collected in a container which was prefilled with 30 uL of a somatic cell releasing agent (0.10%>Nonoxynol-9 solution Neonol AF9-10, Elarum Corporation), a non-ionic surfactant which selectively lyses non-microbial cells (somatic cells), and 20 uL of an ATPase enzyme (40 uM Apyrase, Sigma #2648) capable of eliminating any endogenous ATP. Intermittent perturbation was applied to solution in collection. A specially prepared container. contains exposed to a dipstick which has been coated with specially prepared sintered and charged glass or sintered plastic material provided by Porex corporation (HDPE 130 mm) and Ace Glass (7176-52) which selectively attracts bacteria, but not somatic cells. Another approached tested involved the coating of the dipstick with the binding domains from Strep and Staph phage lytic enzymes prepared by New Horizons Diagnostics, Inc. (NHD). The coated dipstick is so designed that it contains a large attractive surface as shown in figure (E). The dipstick was submerged in the 2 ml sample of milk and allowed to remain in contact with solution for various time periods ranging from 1 to 3 minutes. After the request time, the dipstick, with the captured bacteria, was then removed from the sample collection container and placed in second container containing 100 uL of an ATPase enzyme (20 uJVI Apyrase, Sigma #2648) capable of eliminating any endogenous ATP that may be carried forward from the first step. After one minute, the dipstick, with the captured bacteria, is then removed from the second container and placed in reading cassette which contains a 25 uL of an ATPase inhibitor 0.02% to 1% cetyl trimethylammonium bromide (CTAB, Sigma Chemical), 25 uL of a bacterial releasing reagent (BRA, NHD #BRA200) and 50 uL of Promega luciferin-luciferase reagent (containing 25 mM HEPES buffer (pH 7.5), 40 pg luciferase, 100 pM luciferin, and 10 mM MgSO4). After addition of dipstick into reading cassette contents were stir for approximately 3 to 5 seconds with dipstick. Immediately after mixing, bioluminescent measurements were performed using a Hygiena Ensure luminometer and all readings were blank corrected.

### Example 9

Determination of the Number Bacteria in a Liquid Biological Sample Using a Combination of Vertical and Lateral Flow Device.

In an effort to enumerate the number of bacteria in biological sample one often encounters interference of endogenous matrix elements such as somatic cells and ATP. In order to use bioluminescence to enumerate the bacteria in the sample one must take all precautions to prevent interference from background ATP concentrations that are independent of the bacterial sources. In one procedure, 100 ul of urine (human) was collected in a container which was prefilled with 15 uL of a somatic cell releasing agent (0.10%>Nonoxynol-9 solution Neonol AF9-10, Elarum Corporation), a non-ionic surfactant which selectively lyses non-microbial cells (somatic cells), and 10 uL of an ATPase enzyme (20 uM Apyrase, Sigma #2648) capable of eliminating any endogenous ATP. Intermittent perturbation was applied to solution in collection container.

After 2 minutes the sample solution is transferred in reading cassette (shown in Figure A). As the sample solution travels through the cassette it encounters a first pad (2) impregnate with 25 uL of an ATPase inhibitor (0.02% to 1% cetyl trimethylammonium bromide (CTAB), Sigma Chemical), a second pad (3) impregnated with 25 uL of a bacterial releasing reagent (BRA, NHD #BRA200) and a third pad (4) containing 50 uL of Promega luciferin-luciferase reagent (containing 25 mM HEPES buffer (pH 7.5), 40 pg luciferase, 100 pM luciferin, and 10 mM MgSO4) freeze dried on a Polyvinyl Alcohol Pad (PVA #15 2.5 Medtronic, Inc.).

The rate of flow of the sample is controlled by the nature of each pad's composition, its length and thickness. After approximately 1 to 2 minutes the Bioluminescent reaction begins and measurements are recorded on an Awareness Technologies Inc. luminometer (model #MPPC 1). All readings were blank corrected using sterile water as the sample. Data was collected by a program provided by ATI.

Sample initially collected in a vessel containing somatic cell releasing agent and Apyrase as indicated in paragraph above.

Acceptable membranes for the devices are:

LIL Membranes: all PVA polyvinyl alcohol

Merocel—Medronics Inc. Mystic CT.

### Examples

CF 90 Low-Density 2 mm AND 5 mm

CF 120 Medium-High Density 1.5 mm, 2.75 mm 5.0 mm

CF150 High Density 2.5 mm AND 5.00 mm

All Other Membranes in diagrams any of those below

1—Porex Corp—filtration group—Fairborn, Ga.

Porex 34436

Porex 4903

Porex 4599

2—Amersham—GE Health, Albany, N.Y.

Amersham Protron

Amersham Hybond

3—Whatman—GE Health

PTFE

PES

1-28. (canceled)

29. A method for determining amounts of bacteria in a liquid sample comprising the steps of:
i) filtering the liquid sample through a filter whose porosity is such that bacteria and somatic cells remain resident on the filter to give a residue on the filter;
ii) treating the retained residue with a non-ionic surfactant which lyses non-microbial cells (somatic cells) selectively, thereby removing their ATP from the residue on the filter by application of positive pressure;
iii) treating the retained microbial residue with a bacterial releasing agent capable of lysing bacterial cells to give a released ATP second residue;
iv) treating the released ATP second residue with a Luciferin/Luciferase reagent to give a third solution; and
v) quantifying bacteria in the third solution by luminescence; with the proviso that the liquid sample is not bovine milk.

30. The method according to claim 29, wherein the liquid sample is obtained from a surface using a cloth, gauze, swab, wipe, non-woven fiber or sponge or collected in a container.

31. The method according to claim 29, wherein the liquid sample is a biological liquid sample.

32. The method according to claim 31, wherein the biological liquid sample is chosen from the group consisting of blood, plasma, serum, uterine fluids, lactation products, amniotic fluids, sputum, saliva, urine, semen, cerebrospinal fluid, bronchial aspirate, bronchial lavage aspirate fluid, perspiration, mucus, liquefied stool sample, synovial fluid, peritoneal fluid, pleural fluid, pericardial fluid, lymphatic fluid, tears, tracheal aspirate, a homogenate of a tissue specimen, or any mixtures thereof.

33. The method according to claim 31, wherein the liquid sample is a fluid or specimen obtained from an environmental source selected from a fluid or specimen obtained or derived from food products, food produce, poultry, eggs, meat, fish, beverages, dairy product, water (including wastewater), ponds, rivers, reservoirs, swimming pools, soils, food processing and/or packaging plants, agricultural places, hydrocultures (including hydroponic food farms), pharmaceutical manufacturing plants, medical facilities, hospitals, nursing homes, rehabilitation facilities, animal colony facilities, or any combinations thereof.

34. The method according to claim 32, wherein the liquid sample is urine.

35. The method according to claim 34, wherein the liquid sample is human urine.

36. The method according to claim 29, wherein the non-ionic surfactant is chosen from the group consisting of Neonol AF9-10 (Nonoxynol-9), saponin, amphipathic glycosides Triton X-100 and Lubrol.

37. The method according to claim 29, wherein the bacterial releasing agent is a bacteriophage lytic enzyme (endolysin) or modified lytic enzyme (genetic or chimeric), quaternary amines, ionic and or non-ionic surfactants.

38. The method according to claim 37, wherein the ionic surfactant is selected from the group consisting of anionic surfactants cationic surfactants and zwitterionic surfactants.

39. The method according to claim 37, wherein the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

40. The method according to claim 37, wherein the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

41. The method according to claim 39, wherein the ionic surfactant is selected from DTAB, CTAB and BDDABr.

42. The method according to claim 37, wherein the zwitterionic surfactant is chosen from the group consisting of sulfobetaine-3-10.

43. The method according to claim 36, wherein the bacteriophage lytic endolysin is selected from Lysostaphin, LysK, Lyse5h, LambdaSa2, OSH3b, and KSN383, LysA, LysA2, LysgaY, truncated lambda Sa2, HSCHAP_Lyso, LysoM23-H5CHAP-OSH3b, and PlyC.

44. The method according to claim 29, wherein the Luciferin/Luciferase reagent is chosen from the group consisting of Hygiena ATP Biomass Kit #CCK4, Promega Bright Glo system and any formulations which contain naturally occurring or genetically recombinant Luciferase.

45. The method according to claim 29, wherein the bacteria are gram positive or gram negative bacteria.

46. The method according to claim 44, wherein the gram-positive bacteria are selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

47. The method according to claim 44, wherein the gram-negative bacteria are selected from the group consisting of Escherichia coli, Klebsiella pneumoniae, Pseudomonas aeruginosa, and Proteus mirabilis.

48. The method according to claim 29, wherein the bacteria are selected from the group consisting of Streptococcus agalactiae, Streptococcus dysgalactiae, Streptococcus spp., Staphylococcus aureus, Staphylococcus spp., Escherichia coli, Klebsiella pneumoniae, Pseudomonas aeruginosa, Enterococcus spp., and Proteus mirabilis.

49-127. (canceled)
